(19) United States
(12) Patent Application Publication
(10) Pub. No.: US 2005/0014131 A1
(43) Pub. Date: Jan. 20, 2005

(54) Methods and apparatus for investigating side effects

(75) Inventors: Vadim Kutsyy, Cupertino, CA (US); Eugeni A. Vaisberg, Foster City, CA (US); Daniel A. Coleman, San Mateo, CA (US)

Correspondence Address: BEYER WEAVER & THOMAS LLP, P.O. BOX 778, BERKELEY, CA, 94704-0778, US

(73) Assignee: Cytokinetics, Inc.

(21) Appl. No.: 10/621,821

(22) Filed: Jul. 16, 2003

(51) Int. Cl. C12Q 1/00; G06F 19/00; G01N 33/48; G01N 33/50

(52) U.S. Cl. 435/4; 702/19

(57) ABSTRACT

Methods, apparatus, and computer programs for investigating and characterising side effects of a treatment having an intended or on-target effect on cells are described. The method can include identifying a group of on-target cellular features of the plurality of cells which are affected by the treatment and are related to the on-target effect. A group of off-target cellular features can also be identified which are different to the on-target cellular features and which are also affected by the treatment and which are related to the side effect. A measure of the side effect based on the off-target cellular features can be obtained. The treatment can then be characterised based on the measure of the side effect. A further method involves capturing an image of the population of treated cells and deriving cellular features from the image. An on-target effect signature, which is characteristic of the on-target effect is created from cellular features relating to cellular properties involved in the intended effect. A side effect signature, which is characteristic of a side effect to the on-target effect, is created using cellular features relating to cellular properties not involved in the intended effect. On-target effect and/or side effect metrics are obtained from the signatures which can be used to characterise the treatment.

### FIELD OF THE INVENTION

The present invention relates to methods, apparatus and computer program products for investigating and characterising treatments or stimulus applied to cells. In particular, the present invention allows a fuller characterisation of a treatment or stimulus by evaluating side effects as well as the effect or effects on which the investigation is focussed.

### BACKGROUND OF THE INVENTION

A variety of methods exist for carrying out assays to investigate the effects of a compound or treatment, for example as part of a drug discovery program or as part of a medical investigation. Such investigations tend to be designed so as to focus on a primary effect of the treatment. Such as, what is the effect of the treatment on a specific condition or mechanism of action, or is the treatment efficacious for a specific condition or mechanism of action, or what is the effect of the treatment.

In such investigations, there can be multiple effects caused by the treatment. However, such investigations tend to focus only on the effect that the investigation is intended to elucidate (herein the “on-target effect”). Hence, in some circumstances, while an investigation may indicate that a treatment has no efficacy for a first condition, or is in fact harmful, it is possible that the treatment could have effects other than the on-target effect, that is side effects (herein “off-target effects”) which could be harmful or beneficial. An example of a drug which can have some negative side effects not detected during the drug development or approval stages would be thalidomide, which had harmful effects not related to its on-target effect. Hence some method by which a treatment can be more fully investigated or characterised would be beneficial.

Further, the interaction between a treatment and an organism, for example the human body, can be very subtle and complex. A large variety of factors can be involved in the mechanism and expression of a disease. Hence, a method which can be used to investigate and characterise treatments at a practicable level and which is appropriate for understanding and elucidating the biological processes involved would be beneficial.

Furthermore, owing to the large number of factors that may be involved and the complexity and subtlety of their interaction, a robust method which can be used to systematically acquire a practicable amount of potentially relevant data for analysis and which can provide a more quantitative indication of the various effects of a treatment, rather than a merely qualitative indication of an effect would be beneficial.

The foregoing discussion of the background to the present invention is not acknowledged to be part of the prior art nor within the common general knowledge of a person of ordinary skill in the art. In particular, the appreciation of the drawbacks of present methods of investigating and characterising treatments is not acknowledged to be part of the prior art and has been presented above merely so as to more clearly present the nature of the present invention.

### SUMMARY OF THE INVENTION

The present invention provides in one aspect, methods, apparatus and software for drug discovery, investigating, characterising or classifying treatments applied to cells and for investigating, characterising or classifying the effects and side effects of treatments on cells.

In one aspect of the invention, a method is provided for investigating a treatment applied to cells. The treatment has an on-target effect on the plurality of cells. An on-target cellular feature or group of on-target cellular features is identified. The on-target cellular feature or features can be affected by the treatment. The on-target cellular feature or features can be related to the on-target effect. An off-target cellular feature or group of off-target cellular features are identified. The off-target cellular feature or group of off-target cellular features can be different to the on-target cellular feature or features. The off-target cellular feature or group of off-target cellular features can also be affected by the treatment and can be related to a side effect of the treatment. A measure of the side effect can be determined based on the off-target cellular feature or features.

In another aspect of the invention, a method is provided for characterising a treatment applied to a population of cells. The treatment can have an on-target effect on the population of cells. A first group of cellular features, which have been affected by the treatment, is identified from a plurality of cellular features of the population of cells. The first group of cellular features can be related to the on-target effect of the treatment. A second group of cellular features can be identified from the plurality of cellular features which have been affected by the treatment and which are not related to the on-target effect of the treatment. A first signature characteristic of the on-target effect from the first group of cellular features can be created. A second signature not characteristic of the on-target effect can be created from the second group of cellular features. A first measure derived from the first signature and a second measure derived from the second signature can be evaluated to characterise the treatment.

In another aspect of the invention, a method is provided for characterising a treatment applied to a population of cells. A plurality of cellular features can be derived from a captured image of cells that have been exposed to the treatment. An on-target effect signature can be created, which is characteristic of an on-target effect of the treatment, from a first one of the plurality of cellular features. The plurality of features can relate to cellular properties involved in the on-target effect. A side effect signature is created, which is characteristic of a side effect to the on-target effect, from a second one of the plurality of cellular features. The second one of the plurality of cellular features can relate to cellular properties not involved in the on-target effect. An on-target effect metric derived from the on-target effect signature and/or a side effect metric derived from the side effect signature can be evaluated to characterise the treatment.

Other aspects of the invention include computer program products, computer program code, data structures and computing devices which can provide the various method aspects of the invention.

These and other features and advantages of the present invention will be described below in more detail with reference to the associated drawings.

### BRIEF DESCRIPTION OF THE DRAWINGS

FIG. 1 is a flow chart illustrating at a high level the general method of investigating or characterising treatments according to an aspect of the invention.

FIG. 2 is a flow chart illustrating an embodiment of the general method illustrated by FIG. 1 in greater detail.

FIG. 3 is a flow chart illustrating cell sample preparation activities of the method illustrated by FIG. 2 in greater detail.

FIG. 4 is a flow chart illustrating image capture and processing activities of the method illustrated in FIG. 2 in greater detail.

FIG. 5 is a schematic block diagram of an embodiment of an image capture and image processing system suitable for carrying out some of the activities illustrated in FIG. 4.

FIG. 6 is a process flow chart illustrating an embodiment of a method for determining an on target metric.

FIG. 7 is a process flow chart illustrating an embodiment of a method for determining an off target metric.

FIG. 8 is a plot of on target metrics and off target metrics for a number of treatments applied to a number of cell lines at different dose levels as an example of a graphical method for evaluating treatments.

FIG. 9 is a process flow chart illustrating a further embodiment of a method of characterising a treatment using an off-target metric.

FIG. 10 is a block diagram of a computer system that can be used to implement various aspects of this invention.

### DETAILED DESCRIPTION

Generally, this invention relates to processes and apparatus for use in investigating and characterising the effects of a treatment or stimulus on cells. The methods and apparatus presented in the following can also be used in order to investigate, characterise, or otherwise quantify, an intended effect under investigation and a one or more side effects on cellular behaviour caused by or resulting from the treatment as will be apparent from the following discussion. The invention also relates to computer programs, machine-readable media on which are provided instructions, data structures, etc. for performing the processes of the invention. Features of cell components, which have been derived from captured images of cells, are analyzed in order to provide some measures, or metrics, indicative of the extent to which the treatment caused various biologically relevant effects. These metrics can then be used to help characterise, classify or otherwise categorise a treatment that has been applied to the cells.

The general method includes the analysis of cellular features derived from images captured by an image capture system. Typically an image will be captured of a cell or plurality of cells, depending on the magnification at which the image is captured and certain markers can be used to highlight in the captured image the component of the cell of interest. The term “marker” or “labeling agent” refers to materials that specifically bind to and label cell components. These markers or labeling agents should be detectable in an image of the relevant cells. Typically, a labeling agent emits a signal whose intensity is related to the concentration of the cell component to which the agent binds. Preferably, the signal intensity is directly proportional to the concentration of the underlying cell component. The location of the signal source (i.e., the position of the marker) should be detectable in an image of the relevant cells.

Preferably, the chosen marker binds indiscriminately with its corresponding cellular component, regardless of location within the cell. Although in other embodiments, the chosen marker may bind to specific subsets of the component of interest (e.g., it binds only to sequences of DNA or regions of a chromosome). The marker should provide a strong contrast to other features in a given image. To this end, the marker should be luminescent, radioactive, fluorescent, etc. Various stains and compounds may serve this purpose. Examples of such compounds include fluorescently labeled antibodies to the cellular component of interest, fluorescent intercalators, and fluorescent lectins. The antibodies may be fluorescently labeled either directly or indirectly.

As part of the general method, the effect of a stimulus or treatment on cells can be investigated using the algorithms and processes described herein. The term “treatment” or “stimulus” refers to something that may influence the biological condition of a cell. Often the term will be synonymous with “agent” or “manipulation.” Stimuli may be materials, radiation (including all manner of electromagnetic and particle radiation), forces (including mechanical (e.g., gravitational), electrical, magnetic, and nuclear), fields, thermal energy, and the like. General examples of materials that may be used as stimuli include organic and inorganic chemical compounds, biological materials such as nucleic acids, carbohydrates, proteins and peptides, lipids, various infectious agents, mixtures of the foregoing, and the like. Other general examples of stimuli include non-ambient temperature, non-ambient pressure, acoustic energy, electromagnetic radiation of all frequencies, the lack of a particular material (e.g., the lack of oxygen as in ischemia), temporal factors, etc.

Specific examples of biological stimuli include exposure to hormones, growth factors, antibodies, or extracellular matrix components. Or exposure to biologics such as infective materials such as viruses that may be naturally occurring viruses or viruses engineered to express exogenous genes at various levels. Biological stimuli could also include delivery of antisense polynucleotides by means such as gene transfection. Stimuli also could include exposure of cells to conditions that promote cell fusion. Specific physical stimuli could include exposing cells to shear stress under different rates of fluid flow, exposure of cells to different temperatures, exposure of cells to vacuum or positive pressure, or exposure of cells to sonication. Another stimulus includes applying centrifugal force. Still other specific stimuli include changes in gravitational force, including sub-gravitation, application of a constant or pulsed electrical current. Still other stimuli include photobleaching, which in some embodiments may include prior addition of a substance that would specifically mark areas to be photobleached by subsequent light exposure. In addition, these types of stimuli may be varied as to time of exposure, or cells could be subjected to multiple stimuli in various combinations and orders of addition. Of course, the type of manipulation used depends upon the application.

As part of the processing of captured images, certain features of the cells can be extracted using suitable image processing techniques. The algorithms and processes of the present invention can take this feature data as input in order to carryout their analysis. As used herein, the term “feature” or “cellular feature” refers to a property of a cell or population of cells derived from cell images and includes the basic “parameters” extracted from a cell image. The basic parameters are typically morphological, concentration, and/or statistical values obtained by analyzing a cell image showing the positions and concentrations of one or more markers bound within the cells. Examples of the various features used by the algorithms and processes are given later on herein. It will be appreciated in the following that the algorithms and processes of some aspects of the present invention can work directly from the feature data, and may not need to themselves process the images from which the feature data has been obtained. In other embodiments, the algorithms may processes images in order to obtain information.

Generally, a wide number of cell components can be detected and analyzed. Cell components can include proteins, protein modifications, genetically manipulated proteins, exogenous proteins, enzymatic activities, nucleic acids, lipids, carbohydrates, organic and inorganic ion concentrations, sub-cellular structures, organelles, plasma membrane, adhesion complex, ion channels, ion pumps, integral membrane proteins, cell surface receptors, G-protein coupled receptors, tyrosine kinase receptors, nuclear membrane receptors, ECM binding complexes, endocytotic machinery, exocytotic machinery, lysosomes, peroxisomes, vacuoles, mitochondria, Golgi apparatus, cytoskeletal filament network, endoplasmic reticulum, nuclei, nuclear DNA, nuclear membrane, proteosome apparatus, chromatin, nucleolus, cytoplasm, cytoplasmic signaling apparatus, microbe specializations and plant specializations.

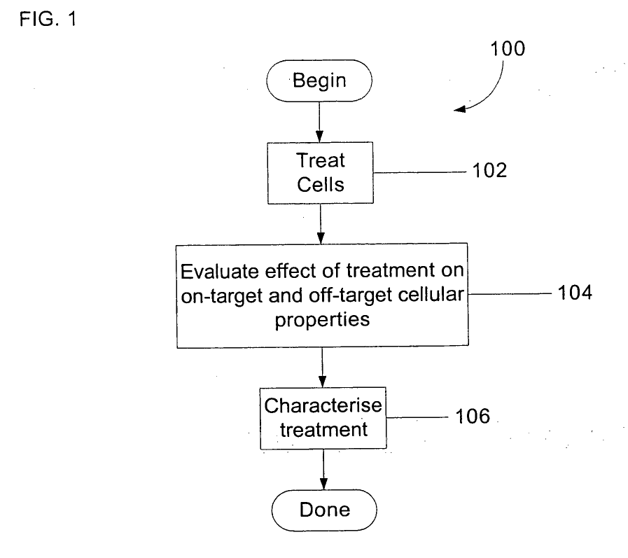

With reference to FIG. 1, there is shown a flow chart 100 illustrating, at a high level, a general method of investigating or characterising a stimulus or treatment that has been applied to cells. As indicated above, the treatment or stimulus applied to the cells can take many forms. In an embodiment of the invention, the treatment can be in the form of a chemical compound, for example a potential or candidate pharmaceutical or drug. The treatment can have a known or an intended effect, or an effect which it is intended to investigate, upon the cells. For example the treatment can be intended to affect a particular biological process or cellular component of the cells, or the investigation can be intended to determine how or whether the treatment affects a particular biological process or cellular component or components. The intended effect can already be known, through previous assays of the treatment, or alternatively, an investigation can be an initial one in which an intended effect on the cell is known, e.g. mitotic arrest, but the extent to which the treatment results in that effect may be unknown. Nonetheless, there is some first or intended effect on the cells which the treatment has, is believed to have or may have. This intended effect will also be referred to herein as the “on-target” effect and generally means an expected or intended effect under investigation for the treatment on cells. The on-target effect need not be the dominant effect of the treatment on the cells but is the effect targeted for investigation.

At step 102 a population, or populations, of cells is exposed to the treatment or stimulus according to any suitable experimental protocol. The cell may be treated using a chemical agent which can be any type of chemical or chemical compound and may in particular be a potential drug or pharmaceutical, any other type of therapeutic agent. Typically, a chemical agent may be delivered in a solution and/or with other compounds or treatments, and at varying dose levels. The cells may also be exposed to a biological treatment, such as a virus, protein or by having the cells' DNA modified by any other means by which biological effects may be induced in the cells. An example of an experimental protocol will be described later in greater detail.

An experiment into the effect of a treatment can typically be carried out by combining sets of assay plates to achieve some scientific purpose. An assay plate is typically a collection of wells arranged in an array with each well holding at least one cell or a related group or population of cells which have been exposed to a treatment or which provides a control group, population or sample. In other embodiments, multiwell plates are not used and single sample holders can be used. As explained above, a treatment can take many forms and in one embodiment can be a particular drug or any other external stimulus (or a combination of stimuli and/or drugs) to which cells are exposed on an assay plate or have previously been exposed. Experimental protocols for investigating the effect of a treatment will be apparent to a person of skill in the art and can include variations in the dose level, incubation time, cell type, cell line and other parameters which are typically varied as part of an experimental protocol.

After the cells have been treated, the extent of the effect of the treatment for the on-target effect is evaluated in step 104. The evaluation of the extent to which the treatment affects the on-target effect is determined by investigating, in a quantitative way, how the properties of the cells that are involved in or related to the on-target effect have changed.

For example, the on-target effect could be mitotic arrest in which case the efficacy of a treatment in delaying the progression of mitosis, or arresting cells in mitosis, could be under investigation. After the treatment has been applied to the cells and features have been extracted from captured images, then some of the cellular features can be used to classify cells as interphase or mitotic. For example, the amount of fluorescence from an anti-phospho-histone 3 (PH3) coupled to a fluorophore can be used to distinguish between mitotic and interphase cells. If PH3 staining is not available, or desirable, then cells can be classified as mitotic or interphase based on a combination of the size of nuclei and the amount of DNA material in nuclei (as revealed by DNA staining using DAPI or Hoechst stains). Mitotic cell DNA is generally smaller and brighter (i.e. captured images have higher mean and median pixel intensities) than DNA in interphase cells. Although there is no real nucleus during mitosis in mammalian cells, amounts of DNA can still be identified. After each cell, or image object, has been classified as interphase or mitotic (or discarded as being an imaging artefact), the proportion of mitotic cells in the cell population can be calculated and provides a metric for the on-target effect: in this example a mitotic index. The effect of the treatment can then be determined by comparing the mitotic index for the treated cells with the mitotic index for a control group of cells. An increase in the mitotic index compared with the negative controls is an indication that the treatment promotes mitotic arrest.

In the above example, mitotic arrest of cells is the on-target effect or property, and a cellular feature, or group of cellular features, which are characteristic of that effect are used to indicate the extent of that effect. In the above example, the detection of PH3 is used. Alternatively, in the above example, the size of the nuclei in the cells and/or other features relating to nuclear size can be used as the cellular feature, or group of cellular features, as, in general, mitotic arrest causes nuclei to be smaller than the nuclei of interphase cells. Therefore the size of the nuclei in the treated cells is a cellular feature which is related to the on-target effect of interest. Other cellular features, involved in mitotic arrest, are also cellular features which are related to the on-target effect. For example the nuclear perimeter, nuclear area, nuclear form factor and other metrics relating to the morphology, shape or texture of a nucleus could also be used as cellular features related to the on-target effect.

There will likely be other cellular features of cell components which are involved in or relate to mitotic arrest and which will also be affected by the treatment and so change. Therefore, from the set of all cellular features, there will be a subset which relate to mitotic arrest (the on-target cellular features). Therefore using a one or a combination of the on-target cellular features, the effect of the treatment on the on-target effect can be evaluated.

It is possible that there will be a number of cellular features which will not be affected by the treatment and these can be considered to be “irrelevant” or neutral cellular features as the treatment has no noticeable or substantial affect on them.

As well as producing the on-target effect, the treatment may have a one or a number of side effects or “off-target” effects on the cells. For example, as well as a treatment causing mitotic arrest, the same treatment may also cause the breakdown of the actin cytoskeleton of a cell, or a Golgi apparatus in interphase cells. This breakdown may be a more or a less dominant effect of the treatment than mitotic arrest, but nonetheless it can be considered to be a “side effect” or “off-target effect” as it is not the intended or targeted effect (which in this example is mitotic arrest) of the treatment under investigation.

For any treatment, there will likely be a number of cellular features relating to a cell or cell components which are related to the side or off-target effect or effects. For example cellular features relating to or characteristic of the Golgi apparatus can be used to determine the extent of the off-target effect of the treatment on the proteins involved in the maintenance of the Golgi, and which are not involved in mitotic arrest. Therefore, there will be a number of cellular features which are affected by the treatment, but which are not related to the on-target effect. A one, some or all of those cellular features can be considered off-target cellular features which can be used in step 104 to evaluate the extent of the effect of the treatment on off-target effects.

It is envisaged that there may be one or more side or off-target effects and that different groups of off-target cellular features may be used in order to evaluate or assess the effect of the treatment on the multiple side effects. In some instances, the side effect may be toxicity. However, in general, the side or off-target effects of a treatment can be any effect on the cellular proteins which are not related to the intended or on-target effect under investigation.

By evaluating 104 both the on-target and off-target effects of the treatment, a better characterisation of the treatment on the cells can be obtained at step 106. Conventional, investigations have tended to focus on the single intended effect of a treatment and side effects have not been systematically evaluated in order to better characterise the overall effect of the treatment of the cells. For instance, a treatment may have a high an efficacy as a mitotic arrest agent but may also be highly toxic and result in significant cell death. Therefore, an investigation which evaluates the affect on mitotic arrest alone would not necessarily highlight this important and potentially harmful side effect. Therefore, the methods of the present invention allow a better characterisation of the overall affect of the treatment by considering the intended effect and also evaluating side effects.

Further, it has been found that different dose levels and experimental protocols can result in different levels at which the intended and side effects occur. Therefore, a treatment, which under conventional investigation methods may be discarded from further evaluation as being either harmful or non-efficacious, can be identified as beneficial under methods of the present invention. Also appropriate dose levels can be determined at which the desired effects are increased and the harmful effects are reduced, which otherwise would not be identified in the absence of information as to the extent of any side effects. Therefore at step 106, the treatment can be characterised based on the on-target effect and any off-target effects, and, in some embodiments, over multiple experimental conditions. It will be appreciated that the on-target effect is not limited to being a beneficial effect and can be a beneficial or harmful effect on the cells, and similarly the off target effect is not limited to being a harmful effect and may also be beneficial or harmful, depending on the context of the overall investigations.

Having discussed the overall methodology of the invention, an example embodiment will now be described in greater detail in the context of an image based collection of cellular features and using the example of mitotic arrest. However, it will be understood that the invention is not limited to investigation of the effect of a treatment on mitotic arrest and side effects thereof, but is applicable to any treatment and to any effect on cellular components, mechanisms or activities and side effects. In particular, the on-target cellular features, relating to the on-target effect, and the off-target cellular features, relating to the off-target effect, will be entirely application dependent. The off-target and on-target cellular features will depend on a number of factors, including: the nature of the intended on-target effect of the treatment and of any anticipated side effects; specific assay configurations, such as cell lines and markers used in the assay; the desired sensitivity; the concentration or dose levels of the treatment; the definition of the on-target and off-target effects; and the sensitivity of the assay at detecting the off-target effects.

Different types of cells can be used in the investigations. For example, for side effects of anti-mitotic cancer treatments, a set of transformed and primary cell lines can be used. Cell lines or mixed cell cultures that can serve as a surrogate for specific types of toxicity can be used, for example primary hepatocytes or hippocampal neurons.

Cellular features relating to various different types of generic cellular phenomena can be related to the on-target and off-target effect, such as changes in growth rate, cell cycle status, cytoskeletal organization, cell shape, alterations in organization and functioning of the endocytic pathway, changes in expression and/or localization of transcription factors, receptors and similar.

It is not necessary to know the off-target cellular features in advance as the off-target features are essentially the features which are affected by the treatment but which are not related to the intended or on-target effect of the treatment. Therefore the cellular features to be used in order to evaluate the extent of the off-target effect may only become apparent after the investigations have been initiated. The off-target cellular features may be selected based on biological knowledge of already known potential effects, in which case the investigation it can be determined whether the particular treatment gives rise to any of these effects as a side effect. In another embodiment, computational techniques can be used in order to identify off-target cellular features, if a good training set from previous experiments is available.

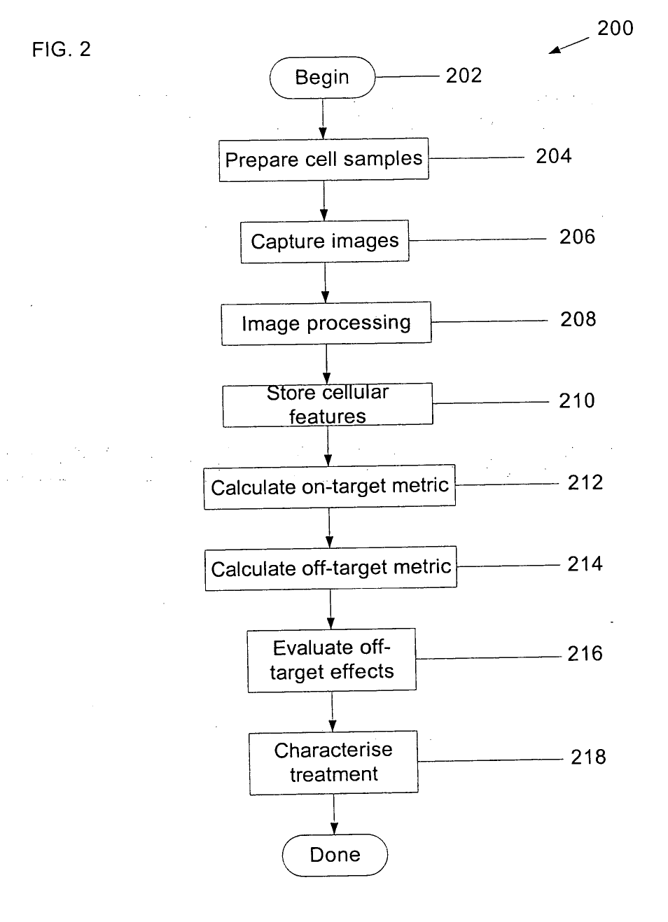

FIG. 2 shows a flow chart 200 illustrating an example of the general method and illustrating various aspects of the invention. The method begins at 202 and at step 204 cell samples are prepared for investigation.

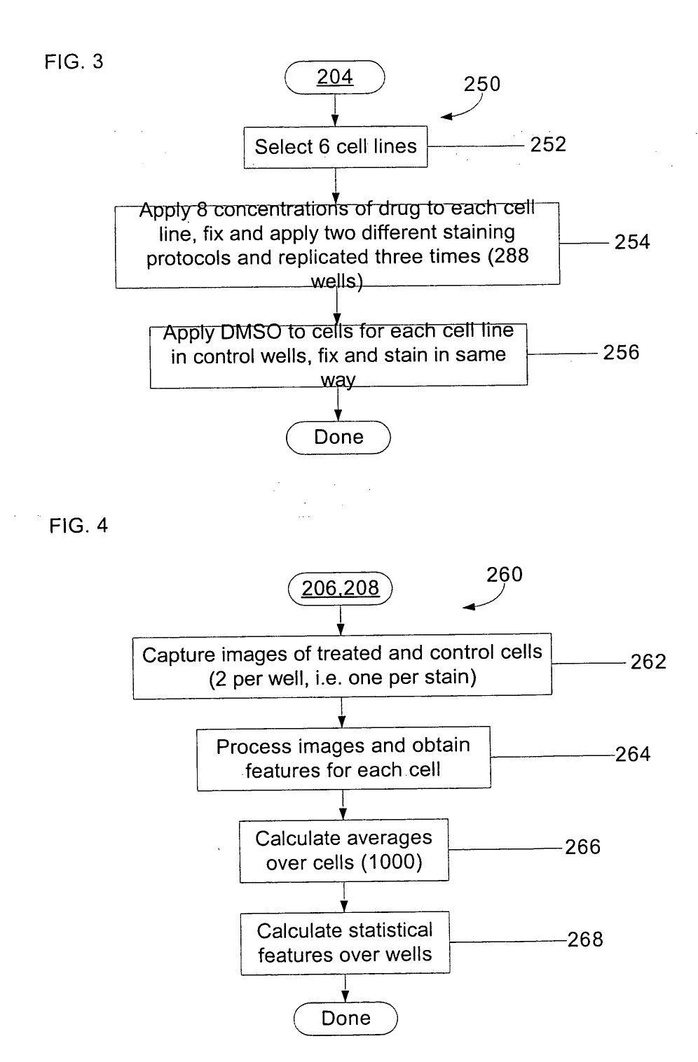

FIG. 3 shows a flow chart 250 illustrating a number of cell sample preparation steps that can be carried out in one embodiment, giving an example of one suitable experimental protocol, and corresponding generally to step 204. Not all the activities and operations illustrated in FIG. 3 are essential. Some operations may be omitted and other operations may be added. The details of each operation may be varied depending on the particular experiment being carried out. For example both off-target and on-target cell features can be obtained from the same marker or stain and multiple staining protocols are not necessary.

Although illustrated as sequential in FIG. 3, steps 254 and 256 do not need to be carried out in sequence and can be carried out in parallel, independently of each other. In a first step 252, a particular cell type is selected and a one or a plurality of different cell lines for that cell type are selected. In the embodiment described, six cell lines for the particular cell type are selected although fewer or more cell lines can be used. In one embodiment, the cell lines used are A549, A498, DU145, HUVEC, SKOV3 and SF268. At step 254, the treatment is applied to the cells. Well plates can be used to hold the cells and a population of cells from a single cell line is provided in each separate well arranged over a well plate or a number of well plates.

In the illustrated embodiment, at step 254, the cells are treated, chemically fixed, stained and placed in wells. However, this is not necessary and in another embodiment, live cells can be used which express a fluorescent protein or stained with live dyes and so no fixing or staining operations are required. In greater detail, wells are provided holding a population of cells. The treatment, in this example a compound, to be investigated is applied to the cells at different concentration levels, by dilution in culture medium. In this example, eight different concentration or dose levels are used, with a different dose level in each well. Fewer or more dose levels can be used as appropriate. The experiment is replicated three times so as to provide three sets of results for each concentration level. Fewer replicates can be used based on cost considerations, but larger numbers of replicates are preferred as providing data with a lower noise level. The drug and cells can be allowed to incubate for a fixed period of time, e.g. in one embodiment 24 hours, to allow the treatment to take effect. In other embodiments, the cells are allowed to incubate for varying periods of time, in order to investigate the time variation of the treatment. The cells can then be chemically fixed, for a single time point assay. The cells for each cell line are subject to a first staining protocol and a second staining protocol, which may involve multiple stains depending on the number and type of cellular features to be marked. Hence, in the described embodiment, 288 wells (eight dose levels, six cell lines, two staining protocols and three replicates) are used each holding a cellular population or group therein.

At the same time as the treated cells are being prepared, a number of control populations of cells are also prepared in step 256. The cells are subject to the same staining treatments, fixation and incubation periods as the treated cells, but without being subjected to the treatment. In one embodiment, the cells are incubated with DMSO, at the same concentrations levels as that used to administer the treatments, in order to provide controls for each cell line and staining or experimental condition. In one embodiment eight control wells are provided on each well plate. This provides at least one control for each cell line/staining protocol combination. Hence the cell sample preparation step 204 results in eight treatment concentrations, in triplicate, with cells stained according to two different protocols, and for six different cell lines and with control populations of cells which have not been exposed to the treatment. It is not necessary to use more than one stain or staining protocol and in other embodiments a single stain only can be used.

Returning to FIG. 2, the cellular features can be obtained from the cells using an image capture and processing technique. At step 206, images of the cells are captured and at step 208 various imaging processing operations are carried out and cellular features are derived from the captured images of the cells. Once all the desired the cellular features have been obtained from the images, or derived from other cellular features, then the cellular features are stored for future use in the evaluation of the on-target and off-target effects at step 210. In another embodiment, the cellular features are used straight away to compute the on-target and off-target effects and then discarded.

FIG. 4 shows a flow chart 260 illustrating the image capture 206, processing and feature extraction 208 steps of flow chart 200 in greater detail. At a first step 262, images of the cell populations in each well are captured. . Images are captured for each of the eight concentration levels, in triplicate for each cell line and for both of the staining protocols. Similarly, images are captured for each of the groups of control cells for each cell line and for both staining protocols. In particular, a first image or set of images is captured of each well for the stains used in the first staining protocol and then a second image or group of images for each well is captured for the stains used in the second staining protocol. One or more images can be captured for each well and/or each stain.

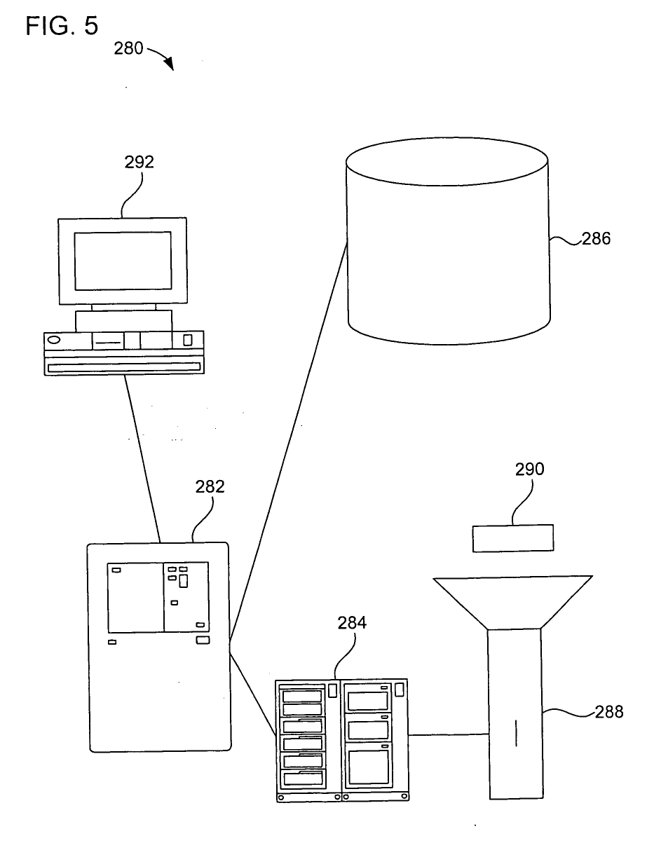

FIG. 5 shows a schematic block diagram of an image capture and image processing system 280 which can be used to capture and process the images of cells or cell parts during steps 206 and 208 and store the cellular features in step 210. This diagram is merely an example and should not limit the scope of the claims herein. One of ordinary skill in the art would recognize other variations, modifications, and alternatives. The present system 280 includes a variety of elements such as a computing device 282, which is coupled to an image processor 284 and is coupled to a database 286. The image processor receives information from an image capturing device 288 which includes an optical device for magnifying images of cells, such as a microscope. The image processor and image capturing device can collectively be referred to as the imaging system herein. The image capturing device obtains information from a plate 290, which includes a plurality wells providing sites for groups of cells. These cells can be cells that are living, fixed, cell fractions, cells in a tissue, and the like. The computing device 282 retrieves the information, which has been digitized, from the image processing device and stores such information into the database 286.

A user interface device 292, which can be a personal computer, a work station, a network computer, a personal digital assistant, or the like, is coupled to the computing device. In the case of cells treated with a fluorescent marker, a collection of such cells is illuminated with light at an excitation frequency from a suitable light source (not shown). A detector part of the image capturing device is tuned to collect light at an emission frequency. The collected light is used to generate an image, which highlights regions of high marker concentration.

Sometimes corrections can be made to the measured intensity. This is because the absolute magnitude of intensity can vary from image to image due to changes in the staining and/or image acquisition procedure and/or apparatus. Specific optical aberrations can be introduced by various image collection components such as lenses, filters, beam splitters, polarizers, etc. Other sources of variability may be introduced by an excitation light source, a broad band light source for optical microscopy, a detector's detection characteristics, etc. Even different areas of the same image may have different characteristics. For example, some optical elements do not provide a “flat field.” As a result, pixels near the center of the image have their intensities exaggerated in comparison to pixels at the edges of the image. A correction algorithm may be applied to compensate for this effect. Such algorithms can be developed for particular optical systems and parameter sets employed using those imaging systems. One simply needs to know the response of the systems under a given set of acquisition parameters.

After the images have been captured, at step 264, the captured images are processed using any suitable image processing and image correction techniques in order to extract the cellular features for the cells from the stored captured images.

A number of image processing steps can be carried out in step 264 and not all the steps described are essential. Certain steps may be omitted and other steps may be added depending on the exact nature of the image capture process and markers used. The image can be corrected to remove any artefacts introduced by the image capture system and to remove any background. Other conventional image correction technique which will improve the quality of the image can also be used. Typically, nuclear markers and cytoplasmic markers generate radiation at different wavelengths and so separate nuclear images and cytoplasmic images may be captured. Therefore different image correction techniques may be used for the nuclear and cytoplasm images, or for images captured of different markers or stains. Similarly, in the rest of the processes, different techniques may be used for the nuclear and cytoplasmic images, depending on the markers used. Also, different processing techniques can be carried out depending on the type of imaging that is used, e.g. brightfield, confocal or deconvolution.

After image correction, a segmentation process is carried out on the images in order to identify individual objects or entities within the image. Any suitable segmentation process may be used in order to obtain various cellular objects or components, such as nuclear and cellular objects and components. Typically nuclear DNA markers provide a strong signal and there is a high contrast in the image and an edge detection based segmentation process can be used. For segmenting cells, a watershed type method can be used instead. The segmentation process typically identifies edges where there is a sudden change in intensity of the cells in the image and then looks for closed connected edges in order to identify an object. Segmentation will not be described in greater detail as it is well understood in the art and so as not to obscure the present invention.

Additional operations may be performed prior to, during, or after the imaging operation 206 of FIG. 2. For example, “quality control algorithms” may be employed to discard image data based on, for example, poor exposure, focus failures, foreign objects, and other imaging failures. Generally, problem images can be identified by abnormal intensities and/or spatial statistics.

In a specific embodiment, a correction algorithm may be applied prior to segmentation to correct for changing light conditions, positions of wells, etc. In one example, a noise reduction technique such as median filtering is employed. Then a correction for spatial differences in intensity may be employed. In one example, the spatial correction comprises a separate model for each image (or group of images). These models may be generated by separately summing or averaging all pixel values in the x-direction for each value of y and then separately summing or averaging all pixel values in the y direction for each value of x. In this manner, a parabolic set of correction values is generated for the image or images under consideration. Applying the correction values to the image adjusts for optical system non-linearities, mis-positioning of wells during imaging, etc.

Generally the images used as the starting point for the methods of this invention are obtained from cells that have been specially treated and/or imaged under conditions that contrast the cell's marked components from other cellular components and the background of the image. Typically, the cells are fixed and then treated with a material that binds to the components of interest and shows up in an image (i.e., the marker).

At every combination of dose, cell line and staining protocol, one or more images can be obtained. As mentioned, these images are used to extract various parameter values of cellular features of relevance to a biological, phenomenon of interest. Generally a given image of a cell, as represented by one or more markers, can be analyzed, in isolation or in combination with other images of the same cell (as provided by different markers), to obtain any number of image features. These features are typically statistical or morphological in nature. The statistical features typically pertain to a concentration or intensity distribution or histogram.

Some general feature types suitable for use with this invention include a cell, or nucleus where appropriate, count, an area, a perimeter, a length, a breadth, a fiber length, a fiber breadth, a shape factor, a elliptical form factor, an inner radius, an outer radius, a mean radius, an equivalent radius, an equivalent sphere volume, an equivalent prolate volume, an equivalent oblate volume, an equivalent sphere surface area, an average intensity, a total intensity, an optical density, a radial dispersion, and a texture difference. These features can be average or standard deviation values, or frequency statistics from the parameters collected across a population of cells. In some embodiments, the features include features from different cell portions or cell lines.

Examples of some specific cellular and nuclear features and parameters that may be extracted from the captured images during step 264 are included in the following table. Other features and parameters can also be used without departing from the scope of the invention.

| Name of Parameter/Feature | Explanation/Comments |
|---|---|
| Count | Number of objects |
| Area | |
| Perimeter | |
| Length | X axis |
| Width | Y axis |
| Shape Factor | Measure of roundness of an object |
| Height | Z axis |
| Radius | |
| Distribution of Brightness | |
| Radius of Dispersion | Measure of how dispersed the marker is from its |
| | centroid |
| Centroid location | x-y position of center of mass |
| Number of holes in closed objects | Derivatives of this measurement might include, for |
| | example, Euler number (=number of objects − number |
| | of holes) |
| Elliptical Fourier Analysis (EFA) | Multiple frequencies that describe the shape of a |
| | closed object |
| Wavelet Analysis | As in EFA, but using wavelet transform |
| Interobject Orientation | Polar Coordinate analysis of relative location |
| Distribution Interobject Distances | Including statistical characteristics |
| Spectral Output | Measures the wavelength spectrum of the reporter |
| | dye. Includes FRET |
| Optical density | Absorbance of light |
| Phase density | Phase shifting of light |
| Reflection interference | Measure of the distance of the cell membrane from |
| | the surface of the substrate |
| 1,2 and 3 dimensional Fourier | Spatial frequency analysis of non closed objects |
| Analysis | |
| 1,2 and 3 dimensional Wavelet | Spatial frequency analysis of non closed objects |
| Analysis | |
| Eccentricity | The eccentricity of the ellipse that has the same |
| | second moments as the region. |
| | A measure of object elongation. |
| Long axis/Short Axis Length | Another measure of object elongation. |
| Convex perimeter | Perimeter of the smallest convex polygon |
| | surrounding an object |
| Convex area | Area of the smallest convex polygon surrounding an |
| | object |
| Solidity | Ratio of polygon bounding box area to object area. |
| Extent | proportion of pixels in the bounding box that are |
| | also in the region |
| Granularity | |
| Pattern matching | Significance of similarity to reference pattern |
| Volume measurements | As above, but adding a z axis |
| Number of Nodes | The number of nodes protruding from a closed |
| | object such as a cell; characterizes cell shape |
| End Points | Relative positions of nodes from above |

After the features have been extracted 264 from the image they are stored 210 in database 286, and analysis of the features is carried out in order to assess the effect of the treatment on the cells.

As explained above, some of the cellular features obtained for the cells are simple features, e.g the area of a nucleus. Other cellular features are statistical in nature, e.g. the standard deviation of the nuclear area for a group of cells, and reflect properties of the group of cells in a well or related wells. It will be appreciated that any simple or complex cellular feature than can be derived from the images is suitable for use in the present invention and that the invention is not to be limited to the specific examples given, nor to the specific sequence of actions, which is merely by way of an illustrative example. The result of step 264 can be thousands or tens of thousands of cellular features derived from each of the treated wells and control wells.

In general in steps 266 and 268 cells from a well are evaluated and some statistics for that well, e.g. the average of a property, are calculated. Then, the same quantity is obtained for the replicate wells (i.e. the other five wells when the experiment is replaicte six time) statistics are computed on those statistics for the replicate wells in order to aggregate (e.g. obtain the median of the average value mentioned above). However, averaging is not necessary and instead cell level information can be used, and have all further computations to be based on cell level information. Hence, for each drug/cell line/time point/marker set/etc there would be thousands of data points. Models based on this would be more complicated and would require greater computing power, but it may provide better estimates compared to the matrix discussed below.

At step 266, at each dose level and for each cell line, the cellular features can be averaged, e.g to obtain an average nuclear area for the cells from a certain cell line at a certain dose level. Hence an average simple cellular feature can be obtained for each cell line at each dose level. However, it is not necessary to calculate averages over cells. Also, other statistical measures can be used such as the median, specific quantiles, standard deviations and other measures of the statictical properties of a group of objects. Further, the statistical properties need not be calculated over all cells, but can be calculated over a sub-population of cells, for example over the sub-group of interphase cells. In that case, a cell cycle related classification of the cells is carried out prior to summarizing or avegaring the cell feature values. For example, in the example where the on-target effect is mitotic arrest, the off target cellular features are computed only for the sub-population of interphase cells, e,g, the average cell area for all interphase cells and not for all cells.

At step 268, more complex cellular features, based on a statistical analysis of the properties of the cells in the wells, rather than the properties of a single cell, are calculated over all the wells for each cell line at each dose level. Hence the cellular features obtained characterise the simple cellular features and statistical cellular features for the cellular populations at each dose level for each cell line.

In other embodiments, the simple cellular features and the statistical cellular features can be determined across cell lines so as to be characteristic of the effect of the treatment across different cell lines. In other embodiments, different incubation times can be used for a given concentration and the cellular features can be averaged over the different incubation times in order to provide cellular features characteristic of the effect of the treatment at the same dose level but over different incubation times.

Returning to FIG. 2, after the cellular features have been calculated and stored, at step 212 a quantitative measure (“on-target metric”) of the extent of the on-target effect is calculated based on the cellular features relating to the on-target effect. In the current example, the on-target effect is mitotic arrest and therefore some metric indicating the extent of mitotic arrest for the cell lines at different dose levels is calculated at step 212. As indicated previously, a wide range of on-target or intended effects can be investigated and the exact nature of the metric will depend on the effect under investigation. However the metric can be derived using the cellular features which are involved in the effect under investigation and which are affected by the treatment. Although steps 212 and 214 are shown sequentially they do not need to be carried out in sequence and are independent of each other and so can be carried out in any order or in parallel.

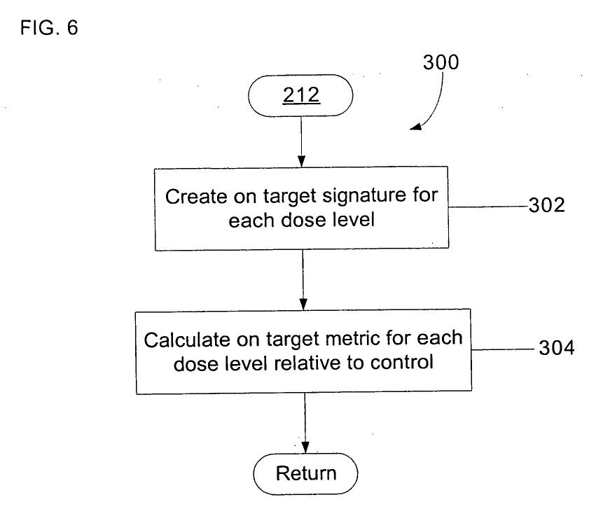

FIG. 6 shows flow chart 300 illustrating in greater detail the operations carried out in calculating the on-target metric and corresponds generally to the method step 212 in FIG. 2. At step 302, the group of cellular features which relate to the on-target effect are identified so as to provide a characteristic signature for the target effect in the cellular population. In the present example, all those cellular features which are indicative of mitotic arrest taking place in a cell are identified and in combination provide the on-target signature. The combination of cellular features providing the on-target signature will be the same for each dose level and each cell line. For example, in identifying mitotic cells, the cellular features used include properties of the nucleus of the cells. As explained above, as one example, the amount of fluorescence from an anti-phospho-histone 3 polyclonal antibody (PH3) coupled to a fluorophore with an object which has been identified as a nucleus can be used to identify mitotic cells. Alternatively, as another example, a combination of the size and amount of nuclear material (as reflected in the captured intensity from stained nuclear DNA) can be can be used to discriminate between interphase and mitotic cells.

The method then proceeds to calculate at step 304 a quantitative measure of the on-target effect relative to the control cells. In this example, the on-target metric is the proportion of mitotic cells in a cellular population. For example the proportion of mitotic cells for a certain dose level may be of order 30%. As will be appreciated, the reliability of determination of the proportion of mitotic cells will depend on the number of cells present in the population of cells being evaluated. For example a determination of 30% from a population of 1500 cells can be considered to have greater reliability than the proportion obtained from a cellular population of, for example, only 100 cells. Further, the calculation of the on-target metric is carried out relative to the control cell population for the cell line. Again the reliability of the determination of the proportion of mitotic cells in the control well will depend on the number of cells in the control well.

Therefore, in one embodiment, in order to take this effect into account, chi-squared statistics are used. A method for obtaining approximate confidnec intervals for the ratio of two binomial proportions based on two independent binomially distributed random variables is used. A chi-square test is used to test the null hypothesis, that the treated and control cell populations can be considered to come from the same cell population, against the hypothesis that the treated cells and the control cells can be considered to come from different cell populations, and hence that the treatment has had a significant effect. The method is described in greater detail in “Confidence Intervals for the Ratio of Two Binomial Proportions”, Biometrics Volume 40, Issue 2, pp. 513-517, June 1984 which is incorporated herein by reference for all purposes.

In particular, where n is the total number of objects (cells) and X is the number of objects under investigation (i.e. mitotic cells) and with the subscript t referring to treated cells and c referring to control cells, then:
p′=((nₜ+n_{c}+Xₜ+X_{c})−{(nₜ+n_{c}+Xₜ+X_{c})²−4(nₜ+n_{c})(Xₜ+X_{c})}^{1/2})/2(nₜ+n_{c})
under the null hypothesis H₀ θ=1, and the chi squared statistic I is given by:
I=(nₜ(pₜ−p′)²+n_{c}(p_{c}−p′)²)/p′(1−p′)
Where p′ is calculated as given above, and pₜ is the proportion of mitotic treated cells and p_{c} is the proportion of mitotic control cells. Although chi square statistics are used to provide the test, other statistics can be used.

Hence the end result of step 304 is a quantitative measure of the extent of on-target effect of the treatment on the cell line at a particular dose level relative to the control group for that same cell line. As will be appreciated, in other embodiments, the value can be calculated across the cell lines rather than on a per cell line basis. Also, it is not essential to calculate the mitotic index taking into account the properties of the control group in order to arrive at a suitable on-target metric. However, it is preferred if the on-target metric is calculated using on-target cellular features which vary with respect to the control group of cells.

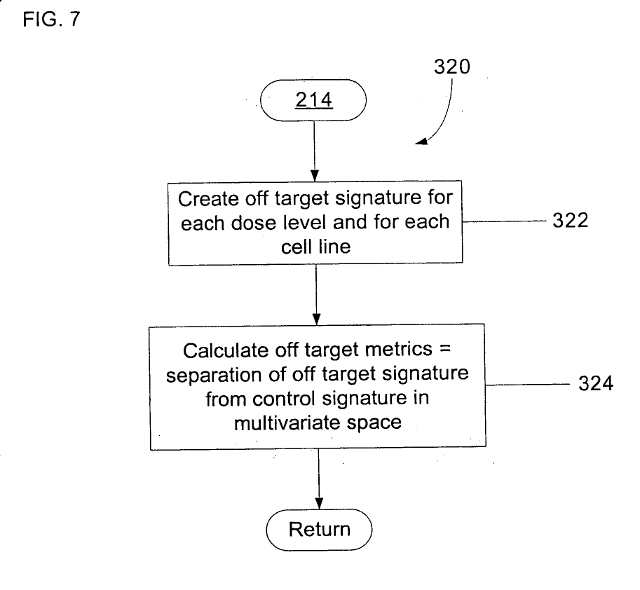

Returning to FIG. 2, at step 214, an off-target metric is defined as will be described in greater detail with reference to FIG. 7. FIG. 7 shows a flow chart 320 illustrating an embodiment of a method for calculating an off-target metric in greater detail and corresponding generally to step 214. At step 322, the group of cellular features which exclude the on-target features and are characteristic of the off-target effect of the treatment on the cell are identified to create a “signature” that is characteristic of an effect on the cell which is different to the on-target effect. For example, in the case of mitotic arrest, any cellular features not relating to mitosis or cell cycle and which are also affected by the treatment can be used. For example cellular features indicative of a cell being in interphase can be used as these cells are not undergoing mitosis. A wide variety of cellular features, as described above and others that will be apparent, can be used. Cellular features can relate to nuclear or cellular morphology, e.g. size, area, shape metrics, branching. Cellular features relating to measures of the total amount of a component of a cell can be used, e.g. the total tubulin, total Golgi apparatus and other measures, often derived from measurements of the total intensity of radiation captured from a particular component of a cell. Also, measures of the texture of a cellular image can be used and which relate to physical properties of components of cells.

More specifically, in the example under discussion, a particular group of off-target cellular features for characterising the off-target effectiveness of a mitotic arrest drug, could include, for all cells that are not mitotic:

(i) the average size of cell nuclei;

(ii) the average elliptical axis ratio for nuclei;

(iii) the average kurtosis intensity of cells;

(iv) the average pixel intensity for Golgi apparatus in cells;

(v) the average cell area;

(vi) the elliptical axis ratio for cells;

(vii) the form factor (area divided by perimeter) for cells;

(viii) the kurtosis of the intensity of tubulin;

(ix) the second moment of a cell;

(x) the average total intensity of tubulin for each cell;

(xi) the proportion of branched (i.e. having projections) cells.

In this example, the above group of cellular features constitutes the group of off-target cellular features which in combination define the off-target signature. A sub-group of these features can be used, or alternatively other groups of off-target cellular features can be used. As will be appreciated, there are a large number of variables in this group of features. Some of these variables may be more important than others, i.e. may be more affected by the treatment than others. The combination of these features can be thought of as defining a vector in a multivariate space (defined by the cellular features) and which is characteristic of the off-target effect, i.e. provides a signature of the off-target effect.

At step 324, a quantitative measure of the extent of the off-target effect is determined by calculating an off-target metric at each dose level and for each cell line. In another embodiment, the off-target metric can be calculated for the combination of all cell lines. The degree to which the treatment causes an off-target effect is reflected in the separation in multivariate space between the off-target signature for treated cells and the off-target signature for the control group of cells.

In one embodiment, each cellular feature can be normalised with respect to the other cells in the group of cells at the particular dose level and for the cell line. Each cellular feature is normalised (f_{N}) by subtracting the average value (fₐᵥ) for the cellular feature over the population of cells from the value (f) and dividing by the standard deviation (σ) for the population of cells as follows: f_{N}=(f-fₐᵥ)/σ.

After each cellular feature has been normalised in this way, and similarly for the control group cellular features, a distance in multivariate space is calculated. For the purposes of simplicity of discussion, if it is assumed that there are only three cellular features (a, b, c) comprising the off-target signature, and where the subscript ‘t’ refers to a feature of a treated cell and the subscript ‘c’ refers to a feature of a control cell, then the distance (L₁) in multivariate space between the off-target signature of the treated cells and off-target signature of the control cells can be calculated as L₁=|aₜ-a_{c}|+|bₜ-b_{c}|+|cₜ-c_{c}|, which provides the off-target metric.

Alternatively, the Euclidean distance (L₂) can be calculated using L₂=√((aₜ-a_{c})²+(bₜ-b_{c})²+(cₜ-c_{c})²) to provide the off-target metric. Other methods of calculating the separation in multivariate space between the treated cell off-target signature and the control cell off-target signature can also be used. Further, in other embodiments of the invention, the on-target metric can be calculated in the same way, using on-target signatures, rather than using the example method described above with reference to FIG. 6.

Returning to FIG. 2, after the on-target and off-target metrics have been calculated, the off-target effects of the treatment are evaluated at step 216. In another embodiment only the on-target metric or only the off-target metric are evaluated. As the off-target metric provides a simple quantitative score for the extent of the presence of the off-target effect in the treated cells, a simple thresholding procedure can be used in order to subsequently characterise the treatment as having a significant or insignificant effect. At step 218, the treatment can be characterised based on both, or either, of the on-target and off-target metrics. For example, if the off-target metric exceeds a threshold value, then the treatment can be characterised as having an unacceptable level of side effects. Similarly, the on-target metric can be thresholded to determine whether the treatment does or does not have a required efficacy in terms of the on-target effect being investigated. The level of the thresholds can be derived from previous or other experiments and can be based on a statistical analysis of the results of other experiments. Similarly, statistical analysis can be used in order to determine the confidence with which the on or off-target metrics can be considered to meet the thresholds or not. The off-target metric cam be used generally to designate compounds as toxic or non-toxic, for example, by comparison with a threshold as described above, or to help to rank or prioritize compounds for further investigation. Also, the off-target metric can be used to try and predict specific clinical toxicities by comparing the off-target metric of a treatment to a knowledgebase of off-target metrics for known toxins.

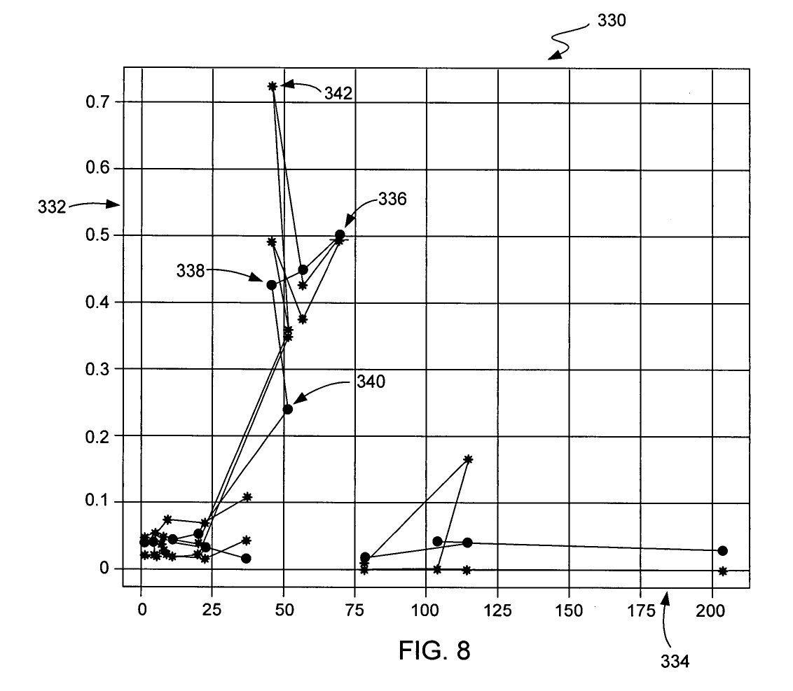

FIG. 8 shows a graphical representation of on-target and off-target metrics for three different treatments and for three different cell lines, by way of illustration of an example of a method of evaluating off-target effects. In particular, FIG. 8 shows a plot 330 of the determined on-target and off-target metrics for three different treatments (two at four different dose levels and one at eight different dose levels) for three different cell lines. The ordinate axis 332 is the on-target metric and the abscissa axis 334 is the off-target metric. This graphical representation of the on-target and off-target metrics provides an example of a method by which the target effects can be evaluated. In this particular example, the on-target metric is a mitotic arrest index.

By way of example of evaluation, point 336 corresponds to a particular dose level for a particular treatment on a particular cell line. As can be seen, at this dose level, both the on-target and off-target metrics are significant. It may be that in the absence of the off-target metric, this dose level would be considered acceptable as providing a desired efficacy with regard to the on-target effect. However, by utilising the off-target metric, this dose level may be identified as being undesirable, e.g. toxic, and so the treatment can be more accurately characterised. Point 338 corresponds to a different dose level for the same compound and the same cell line. At this dose level, the compound may be considered to provide sufficient efficacy and to have sufficiently low off-target effect as to be of utility. In this example, the dose level associated with point 338 is lower than the dose level associated with point 336 and therefore is useful in identifying a suitable dosage level for the treatment in order to avoid unwanted side effects. The dose level correspondent to point 340 is lower than the dose level correspondence to point 338 but at this dose level, the side effects are greater, indicated by the higher off-target metric, and so again this helps to identify dosage levels at which undesirable effects can be reduced.

Similarly, point 342 which corresponds to the same drug as points 336, 338 and 340 but applied to a different cell line shows a high level of on-target effect and possibly an acceptably low level of off-target effect. As can be seen for the dosage levels either side of this point, there is a significant reduction in the on-target effect and also an increase in the off-target effects. Hence the graphical representation of the on-target and off-target metrics can be of use in evaluating the on-target and off-target effects and can provide indications as to further areas of interest to be the subject of further investigations and experiments.

Also, evaluation of the on-target and off-target metrics can be used as a screening method in order to help identify good candidate drugs or pharmaceuticals for further investigation. For example the treatment resulting in the points plotted in the left hand side of the plot may be a better candidate drug than the drug corresponding to the points plotted in the bottom right hand side area of the plot.

With regard to characterising compounds, either the on-target or off-target effect metric reaching a threshold or not reaching a threshold can be used as a mechanism in order to characterise a treatment. For example the set of three lines to the right of the 75 mark on the off-target axis may be considered too harmful for further investigation, if the off-target effect is a harmful one, or alternatively may be considered good candidate compounds if the off-target effect is a beneficial effect. Similarly, the group of lines toward the origin, and which relate to a further treatment, may be considered to indicate that the treatment does not have a sufficient effect on the on-target or off-target effect. However, whether an on-target or off-target metric falls above or below a threshold and so can be considered to be indicative of a useful property, or not, will be entirely application dependent as in some applications exhibiting the effect may be considered beneficial and in other applications not exhibiting the effect may be considered beneficial, and vice versa.

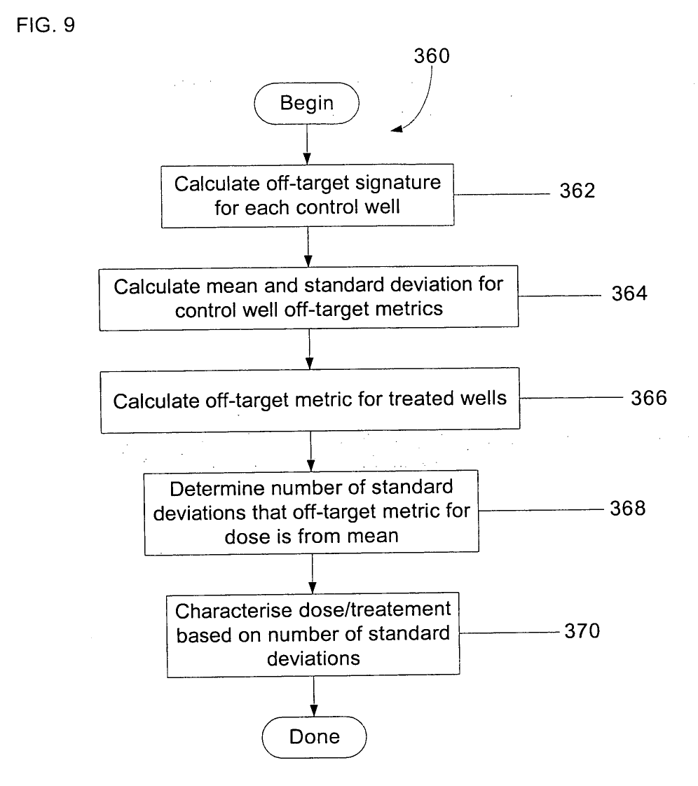

FIG. 9 shows a further method for characterising a treatment based on evaluation of an off-target metric. At step 362, after the group of off-target cellular features have been identified, an off-target metric is calculated for each control well individually. The off-target metric is again a distance in multi-variant space but from the origin of multi-variant space rather than with respect to the control well as described previously. Therefore, using the same nomenclature as before, the distance for a control well can be expressed as L₁=|a_{c}|+|b_{c}|+|c_{c}| and similarly for L₂, with the appropriate changes, and which distance can be used instead in the following.

The distance L₁ is calculated for each control well and then the average distance is calculated together with the standard deviation in step 364. Then the off-target metric for treated wells is calculated at step 366, again relative to the origin of multi-variant space. Then the number of standard deviations between the control well mean off-target metric and the treated well off-target metric is determined at step 368. If the metric for the treated well is considered to lay a significant number of standard deviations from the mean for control wells, then this can be considered indicative of a significant off-target effect and the treatment characterised accordingly at step 370. The actual number of standard deviations that can be considered significant will vary from application to application. For some screens, 10 to 15 standard deviations have been found to be indicative of significance.

Generally, embodiments of the present invention, and in particular the processes involved in the calculation of the on-target and off-target metrics, their evaluation and characterization of the treatments, employ various processes involving data stored in or transferred through one or more computer systems. Embodiments of the present invention also relate to an apparatus for performing these operations. This apparatus may be specially constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, embodiments of the present invention relate to computer readable media or computer program products that include program instructions and/or data (including data structures) for performing various computer-implemented operations. Examples of computer-readable media include, but are not limited to, magnetic media such as hard disks, floppy disks, and magnetic tape; optical media such as CD-ROM disks; magneto-optical media; semiconductor memory devices, and hardware devices that are specially configured to store and perform program instructions, such as read-only memory devices (ROM) and random access memory (RAM). The data and program instructions of this invention may also be embodied on a carrier wave or other transport medium. Examples of program instructions include both machine code, such as produced by a compiler, and files containing higher level code that may be executed by the computer using an interpreter.

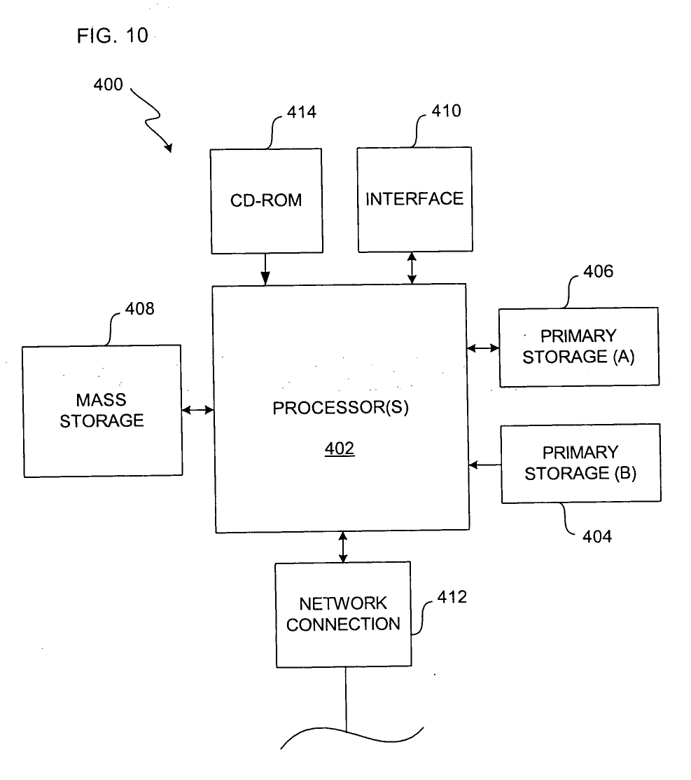

FIG. 10 illustrates a typical computer system that, when appropriately configured or designed, can serve as an image analysis apparatus of this invention. The computer system 400 includes any number of processors 402 (also referred to as central processing units, or CPUs) that are coupled to storage devices including primary storage 406 (typically a random access memory, or RAM), primary storage 404 (typically a read only memory, or ROM). CPU 402 may be of various types including microcontrollers and microprocessors such as programmable devices (e.g., CPLDs and FPGAs) and unprogrammable devices such as gate array ASICs or general purpose microprocessors. As is well known in the art, primary storage 404 acts to transfer data and instructions uni- directionally to the CPU and primary storage 406 is used typically to transfer data and instructions in a bi-directional manner. Both of these primary storage devices may include any suitable computer-readable media such as those described above. A mass storage device 408 is also coupled bi-directionally to CPU 402 and provides additional data storage capacity and may include any of the computer-readable media described above. Mass storage device 408 may be used to store programs, data and the like and is typically a secondary storage medium such as a hard disk. It will be appreciated that the information retained within the mass storage device 408, may, in appropriate cases, be incorporated in standard fashion as part of primary storage 406 as virtual memory. A specific mass storage device such as a CD-ROM 414 may also pass data uni-directionally to the CPU.

CPU 402 is also coupled to an interface 410 that connects to one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 402 optionally may be coupled to an external device such as a database or a computer or telecommunications network using an external connection as shown generally at 412. With such a connection, it is contemplated that the CPU might receive information from the network, or might output information to the network in the course of performing the method steps described herein.

Although the above has generally described the present invention according to specific processes and apparatus, the present invention has a much broader range of applicability. In particular, aspects of the present invention is not limited to any particular kind of treatment, cells, cellular process or assay formats and can be applied to virtually any cellular effects where an understanding of the affect of a treatment on a cell is desired. Thus, in some embodiments, the techniques of the present invention could provide information about many different types or groups of cells, substances, cellular processes and mechanisms of action, and genetic processes of all kinds. One of ordinary skill in the art would recognize other variants, modifications and alternatives in light of the foregoing discussion.

1. A method of investigating a treatment applied to a plurality of cells, the treatment having at least an on-target effect on the plurality of cells, the method comprising:
identifying at least an on-target cellular feature or group of on-target cellular features of the plurality of cells, the on-target cellular feature or features being affected by the treatment and being related to the on-target effect;
identifying at least an off-target cellular feature or group of off-target cellular features different to the on-target cellular feature or features, which are also affected by the treatment and which are related to a side effect of the treatment; and
determining a measure of the side effect based on the off-target cellular feature or features.

2. The method as claimed in claim 1, further comprising characterising the treatment based on the measure of the side effect.

3. The method as claimed in claim 1, further comprising determining a measure of the on-target effect based on the on-target cellular feature or features.

4. The method as claimed in claim 3, further comprising characterising the treatment based on the measure of the on-target effect.

5. The method as claimed in claim 4, further comprising characterising the treatment based on the measure of the side effect and the measure of the on-target effect.

6. The method as claimed in claim 1, wherein the off-target cellular feature or features are not related to the on-target effect.

7. The method as claimed in claim 1, wherein the measure is a distance in a multivariate space corresponding to the off-target cellular features.

8. A method of characterising a treatment that has been applied to a population of cells and that has an on-target effect on the population of cells, comprising:
identifying from a plurality of cellular features of the population of cells, a first group of cellular features which have been affected by the treatment and which are related to the on-target effect of the treatment;
identifying from the plurality of cellular features a second group of cellular features which have been affected by the treatment and which are not related to the on-target effect of the treatment;
creating a first signature characteristic of the on-target effect from the first group of cellular features;
creating a second signature not characteristic of the on-target effect from the second group of cellular features; and
evaluating a first measure derived from the first signature and a second measure derived from the second signature to characterise the treatment.

9. The method as claimed in claim 8, and further comprising: determining the separation in multivariate space between the second signature and an origin.

10. The method as claimed in claim 9, further comprising: determining the separation in multivariate space between the first signature and an origin.

11. The method as claimed in claim 9, wherein the origin is provided by a control signature created from a control group of cellular features of a control group of cells, and wherein the control group of cellular features are the same cellular features as the second group of cellular features.

12. The method as claimed in claim 10, wherein the origin is provided by a control quantitative signature created from a control group of cellular features of a control group of cells, and wherein the control group of cellular features are the same cellular features as the first group of cellular features.

13. A computer program product comprising a machine readable medium on which is provided program instructions for characterising a treatment that has been applied to a population of cells and that has an on-target effect on the population of cells, the instructions comprising:
code for identifying from a plurality of cellular features of the population of cells, a first group of features which have been affected by the treatment and which are related to the on-target effect of the treatment;
code for identifying from the plurality of cellular features a second group of features which have been affected by the stimulus and which are not related to the on-target effect of the treatment;
code for creating a metric characteristic of the on-target effect from the first group of features;
code for creating a second metric not characteristic of the on-target effect from the second group of features; and
code for evaluating the first and second metrics to characterise the treatment.

14. A computing device comprising a memory device configured to store at least temporarily program instructions for characterising a stimulus that has been applied to a population of cells and that has an on-target effect on the population of cells, the instructions comprising:
code for identifying from a plurality of cellular features of the population of cells, a first group of features which have been affected by the treatment and which are related to the on-target effect of the treatment;
code for identifying from the plurality of cellular features a second group of features which have been affected by the treatment and which are not related to the on-target effect of the treatment;
code for creating a first metric characteristic of the on-target effect from the first group of features;
code for creating a second metric not characteristic of the on-target effect from the second group of features; and
code for evaluating the first and second metrics to characterise the treatment.

15. A method of characterising a treatment applied to a population of cells, comprising:
deriving a plurality of cellular features from at least a first captured image of the population of cells that have been exposed to the treatment;
creating an on-target effect signature, which is characteristic of an on-target effect of the treatment on the population of cells, from at least a first one of the plurality of cellular features, the at least one of the plurality of features relating to cellular properties involved in the on-target effect;
creating a side effect signature, which is characteristic of a side effect to the on- target effect, from at least a second one of the plurality of cellular features, the second one of the plurality of cellular features relating to cellular properties not being involved in the on-target effect; and
evaluating an on-target effect metric derived from the on-target effect signature and/or a side effect metric derived from the side effect signature to characterise the treatment.

16. The method as claimed in claim 15, wherein the on-target effect signature is created from a group of cellular features.

17. The method as claimed in claim 16, wherein the side effect signature is created from a further group of cellular features, in which none of the members of the group of cellular features used to create the on-target effect signature and the members of the further group of cellular features used to created the side effect signature are common.

18. The method as claimed in claim 15, wherein the second one of the plurality of cellular features is affected by the treatment.

19. The method as claimed in claim 18, further comprising:
exposing different populations of cells to different doses of the treatment; and
deriving the on-target effect metric and the side effect metric for different doses of the treatment.

20. The method as claimed in claim 15, wherein deriving the on-target effect metric or the side effect metric includes determining the difference between the on-target effect signature or side effect signature and a control signature from the same cellular features for a control group of cells.

21. The method as claimed in claim 15, and further comprising:
capturing at least a first image of a control group of cells; and
deriving a plurality of cellular features from the image of the control group of cells;
creating a control on-target signature for the same cellular features for the control group; and
creating a control side effect signature for the same cellular features for the control group.

22. The method of claim 21, further comprising determining a side effect distance in a multivariate space between the side effect signature and the control side effect signature.

23. The method of claim 22, further comprising determining a target effect distance in a multivariate space between the on-target effect signature and the control on-target effect signature.

24. The method of claim 23, wherein characterising the stimulus is based on the side effect distance.

25. The method of claim 24, wherein characterising the stimulus is based on the on- target effect distance.

26. The method as claimed in claim 25, further comprising generating a graphical representation of the side effect distance and on-target effect distance.
